(19) United States
(12) Patent Application Publication
(10) Pub. No.: US 2025/0249194 A1
(43) Pub. Date: Aug. 7, 2025

(54) NASAL CANNULA FOR SUPPLYING RESPIRATORY GAS TO A PATIENT IN THE CONTEXT OF HIGH-FLOW THERAPY

(71) Applicant: Loewenstein Medical Technology S.A., Luxembourg (LU)

(72) Inventor: Andreas SPECHT, Pfaffenheck (DE)

(21) Appl. No.: 19/036,103

(22) Filed: Jan. 24, 2025

(30) Foreign Application Priority Data
Feb. 1, 2024 (DE) 102024102820.3

(51) Int. Cl. A61M 16/06; A61M 16/00; A61M 16/08

(52) U.S. Cl. CPC A61M 16/0666; A61M 16/0683; A61M 16/0688; A61M 16/0816; A61M 2016/0027; A61M 2240/00

(57) ABSTRACT

A nasal cannula for supplying respiratory gas to a patient in the context of high-flow therapy comprises a nose attachment piece having a hollow main body, a hose connector for connecting a supply hose to the main body, such that respiratory gas can flow from the supply hose into the main body, and two nose pieces projecting from the main body, for conducting the respiratory gas from the main body into the nose of the patient; a first cheek pad for supporting and/or fixing the nose attachment piece on a first cheek of the patient; a second cheek pad for supporting and/or fixing the nose attachment piece on a second cheek of the patient; a retaining band, which can be tensioned around the head of the patient, for applying an additional retaining force to the nose attachment piece and/or the two cheek pads.

### CROSS-REFERENCE TO RELATED APPLICATIONS

The present application claims priority under 35 U.S.C. § 119 of German Patent Application No. 102024102820.3, filed Feb. 1, 2024, the entire disclosure of which is expressly incorporated by reference herein.

### BACKGROUND OF THE INVENTION

### 1. Field of the Invention

The invention relates to a nasal cannula for supplying respiratory gas to a patient in the context of high-flow therapy. The invention further relates to a ventilation system having such a nasal cannula.

### 2. Discussion of Background Information

Nasal high-flow therapy by means of a nasal cannula can be used, for example, in patients suffering from mild to moderate respiratory distress syndrome with acute hypoxemic respiratory failure. In these cases, by means of a continuous flow of fresh gas, the anatomical dead space, i.e. the volume of the airways from the nasal cavity to the terminal bronchioles, which do not participate in the gas exchange, can be utilized as an oxygen reservoir and thus functionally reduced. Nasal high-flow therapy can increase the tidal volume while the respiratory rate decreases. In this way, alveolar ventilation can be improved and respiratory work assisted.

Nasal high-flow therapy is increasingly being used in very small patients such as premature or newborn babies or infants, where it can noticeably improve the success of therapy by comparison with conventional oxygen therapy. In nasal high-flow therapy on these patients, the oxygen flow rate is generally between 1 and 8 l/min.

A corresponding nasal cannula generally comprises a nose attachment piece with two nose pieces (also called prongs) for insertion into the nostrils. The nose attachment piece can be connected to a respiratory gas source via a supply hose, such that respiratory gas can flow with a suitable throughflow into the nose attachment piece and from there onward into the nose via the nose pieces.

Particularly in the case of premature or newborn babies, the attachment or removal of the nasal cannula may be associated with certain difficulties on account of the small head and the particularly sensitive skin.

Furthermore, in the case of nasal high-flow therapy, and for lack of suitable pressure measurement, multiple attempts are often needed before a suitable setting of the ventilation parameters is found.

In view of the foregoing, it would be advantageous to have available an improved nasal cannula for supplying respiratory gas to a patient in the context of high-flow therapy and a corresponding ventilation system.

### SUMMARY OF THE INVENTION

In a first aspect, the invention provides to a nasal cannula for supplying respiratory gas to a patient such as a premature or newborn baby or an infant in the context of (nasal) high-flow therapy. The nasal cannula comprises a nose attachment piece with a hollow main body, a hose connector for connecting a supply hose to the main body, such that respiratory gas can flow from the supply hose into the main body, and two nose pieces projecting from the main body, for conducting the respiratory gas from the main body into the nose of the patient. Furthermore, the nasal cannula comprises a first cheek pad for supporting and/or fixing the nose attachment piece on a first cheek of the patient, a second cheek pad for supporting and/or fixing the nose attachment piece on a second cheek of the patient, and a retaining band which can be tensioned around the head of the patient in order to apply an additional retaining force to the nose attachment piece and/or the two (or at least one of the) cheek pads.

The additional retaining force can be suitable for holding the nose attachment piece in a defined position relative to the patient's face during use of the nasal cannula.

The use of a special retaining band enables particularly secure fixing of the nasal cannula to the very small face of the patient, even when the latter is being very lively. “Nose piece” can be understood to mean a so-called prong for insertion into a single nostril.

“Retaining band” can be understood to mean an elongate element separate from the supply hose, for example a rubber band and/or fabric band or an arrangement of a plurality of such bands.

“Cheek pad” can be understood to mean a flat, particularly flexible element made of an air-permeable and/or liquid-absorbing material, for example a particularly skin-friendly and/or particularly soft textile material. When the nasal cannula is in use, the cheek pads can directly touch the skin of the patient in a planar fashion, such that the contact pressure generated by the additional retaining force, i.e. by the retaining band tensioned around the head, is uniformly distributed over the largest possible surface area of the face, in particular of the cheeks. Undesired pressure points on the face can thus be avoided.

Alternatively or additionally, depending on the embodiment, the nasal cannula may be suitable for at least one of the following patient types: children, young people, adults.

In a second aspect, the invention provides a nasal cannula for supplying respiratory gas to a patient in the content of (nasal) high-flow therapy. The nasal cannula comprises a nose attachment piece with a hollow main body, a hose connector for connecting a supply hose to the main body, such that respiratory gas can flow from the supply hose into the main body, and two nose pieces projecting from the main body, for conducting the respiratory gas from the main body into the nose. Furthermore, the nasal cannula comprises a pressure measurement port for connecting a pressure-measuring device to the nasal cannula, and a pressure measurement line, which is fluidically coupled to the pressure measurement port and, proceeding from the pressure measurement port, extends through the main body into at least one of the nose pieces, in order to enable a pressure measurement in the nose, for example at the outlet of the nose piece or of the nose pieces.

Such a nasal cannula permits a very precise oropharyngeal pressure measurement during high-flow therapy. Thus, ventilation parameters of high-flow therapy—for example respiratory gas pressure, respiratory gas flow, respiratory gas volume or respiratory gas composition-can be controlled and/or evaluated very precisely depending on the measured pressure. This can considerably improve the high-flow therapy compared to embodiments without such direct measurement of the pressure in the nose. Depending on the embodiment, the nasal cannula may be suitable for at least one of the following patient types: premature or newborn babies, infants, children, young people, adults.

The pressure measurement port can be embodied, for example, as a component of a plug connection for connecting the nasal cannula to a respiratory gas source.

By means of the pressure measurement line, the pressure prevailing in the nose or in the nasopharyngeal space can be transmitted to the pressure measurement port separately from the respiratory gas flow in the supply hose. For this purpose, the pressure measurement line can be designed, for example, as a separate hose, a separate tube or a combination of at least one separate hose and at least one separate tube.

It will be noted that the nasal cannula according to one embodiment of the second aspect of the invention can also comprise features of the nasal cannula according to one embodiment of the first aspect of the invention (and vice versa).

In a third aspect, the invention provides a ventilation system. The ventilation system comprises a nasal cannula according to one of the embodiments described above and below, and a supply hose for connecting the hose connector of the nasal cannula to a respiratory gas source, such that respiratory gas can flow from the respiratory gas source via the supply hose into the main body of the nasal cannula. Furthermore, the ventilation system comprises a pressure-measuring device connected to the pressure measurement port of the nasal cannula, for converting a (pneumatic) pressure present at the pressure measurement port into an electrical pressure measurement signal, and a control unit for controlling at least one ventilation parameter of the (nasal) high-flow therapy using the pressure measurement signal.

The control unit can comprise hardware and/or software modules. In particular, the control unit can comprise a processor configured to execute a corresponding (computer-implemented) method for controlling and/or evaluating the ventilation parameters. In addition, the control unit can comprise a memory and/or a data communication interface for wireless and/or wired data communication to peripheral devices. Alternatively, the control unit can be implemented exclusively as hardware, for example in the form of an ASIC component or FPGA component.

The at least one ventilation parameter can comprise, for example, at least one of the following parameters: a respiratory gas pressure, a respiratory gas flow, a respiratory gas volume, a respiratory gas composition.

Such a ventilation system enables particularly precise control of high-flow therapy in comparison with conventional ventilation systems with no direct measurement of the pressure in the nasal cannula.

Various embodiments of the invention are described hereinbelow. These embodiments should not be understood as restricting the scope of the invention.

According to one embodiment, at least one of the components of the nose attachment piece can be manufactured at least partially from silicone.

According to one embodiment, the various components of the nose attachment piece can be produced from the same (plastic) material, for example silicone.

According to one embodiment, the nose attachment piece can be formed in one piece. For example, the nose attachment piece may have been produced in an injection molding process.

According to one embodiment, the nasal cannula can be designed such that the nose attachment piece can be fastened to the retaining band on the one hand via the first cheek pad and on the other hand via the second cheek pad. In other words, the nasal cannula can be designed in such a way that, on the one hand, the retaining band can be fastened (directly) to each cheek pad and, on the other hand, the main body can be fastened (directly) to each cheek pad.

On account of their flat shape, the cheek pads generally offer sufficient space for stable and comfortable fastening of the retaining band. In comparison with the cheek pads, the nose attachment piece is generally quite long and narrow. The possibilities for directly fastening the retaining band to the nose attachment piece are therefore usually limited and are associated with certain compromises as regards stability and/or comfort. By virtue of the fact that the cheek pads are designed as load-bearing connecting elements, such restrictions or compromises can be avoided without great additional outlay.

According to one embodiment, the nasal cannula can be designed such that at least one or each of the cheek pads is or can be fastened to the retaining band and/or to the nose attachment piece via a hook-and-loop connection. This allows comfortable and/or secure fastening in various positions with little structural outlay. Moreover, a connection of this kind can be released very frequently without significant wear occurring. Alternatively or additionally, an adhesive connection is possible for fastening the respective cheek pad to the retaining band and/or to the nose attachment piece.

According to one embodiment, at least one or each of the cheek pads can have, on a first side facing the respective cheek during use of the nasal cannula, a skin-friendly contact section for contacting the respective cheek and, on a second side opposite the first side and facing away from the respective cheek during use of the nasal cannula, a fastening section for fastening the cheek pad to the retaining band and/or to the nose attachment piece. The fastening section can comprise an adhesive portion and/or a fleece portion of a hook-and-loop connection and/or a (self-adhesive) adhesion section for fastening the cheek pad to the retaining band and/or to the nose attachment piece.

According to one embodiment, the contact section can be at least partially composed of silicone and/or comprise a specific adhesive surface for adhesively bonding the respective cheek pad to the respective cheek. A contact section without an adhesive surface has the advantage that no residues of adhesive have to be removed from the skin.

According to one embodiment, at least one or each of the cheek pads can be formed as a layer composite composed of at least a first layer and a second layer, which layers can differ from each other in terms of their material and/or their size (for example their thickness) and/or their shape. Here, the contact section can be a section of the first layer, and/or the fastening section can be a section of the second layer. The second layer can be formed as a carrier for stabilizing the first layer (or vice versa). For example, the carrier can be produced from a (for example particularly air-permeable) textile material.

According to one embodiment, the nose attachment piece can furthermore comprise a first fastening wing projecting from the main body, for fastening the first cheek pad to the nose attachment piece, and/or a second fastening wing projecting from the main body, for fastening the second cheek pad to the nose attachment piece. The first fastening wing can be fastened to the main body in such a way that, during use of the nasal cannula, it projects beyond a first end of the main body toward the first cheek. Alternatively or additionally, the second fastening wing can be fastened to the main body in such a way that, during use of the nasal cannula, it projects beyond a second end of the main body toward the second cheek. For example, the fastening wing or the fastening wings can in each case project at least partially beyond one end of the main body, as viewed in the longitudinal direction of the (elongate) main body. Alternatively or additionally, the fastening wing or the fastening wings can project at least partially laterally beyond the main body.

The fastening wing or the fastening wings can in each case be connected to the main body in a fixed or releasable manner, for example via a hook-and-loop connection and/or an adhesively bonded connection. The fastening wing or the fastening wings can be designed to be particularly flexible in comparison with the main body, in order to allow good adaptation to the facial contours of the patient. In particular, the fastening wing or the fastening wings can be produced from a (for example particularly air-permeable) textile material and/or can have a fleecy surface at least in parts. It is also conceivable for the fastening wing or fastening wings and the main body to be formed in one piece and/or from the same material.

According to one embodiment, the nasal cannula can be designed such that, for fastening the first cheek pad to the nose attachment piece, the fastening section of the first cheek pad can be fastened to the first fastening wing and/or, for fastening the second cheek pad to the nose attachment piece, the fastening section of the second cheek pad can be fastened to the second fastening wing. The respective cheek pad can thus be fastened securely and/or precisely to the nose attachment piece without this appreciably impairing desired properties of the respective cheek pad, such as flexibility or air permeability.

According to one embodiment, the first fastening wing can have a through-opening which connects a top side of the first fastening wing to a bottom side of the first fastening wing opposite the top side. The opening can be arranged in such a way that the fastening section of the first cheek pad, fastened to the first fastening wing, at least partially lies opposite the opening, such that the retaining band can be fastened to the fastening section of the first cheek pad situated opposite the opening.

According to one embodiment, the second fastening wing can have a through-opening which connects a top side of the second fastening wing to a bottom side of the second fastening wing opposite the top side. The opening can be arranged in such a way that the fastening section of the second cheek pad, fastened to the second fastening wing, at least partially lies opposite the opening, such that the retaining band can be fastened to the fastening section of the second cheek pad situated opposite the opening.

In this case, that part of the respective fastening section situated opposite the opening can comprise at least part of the adhesive portion and/or of the fleece portion. In this case, the respective fastening wing can have a further adhesive portion that can be hooked onto the fleece portion, and/or a further fleece portion that can be hooked onto the adhesive portion.

This permits a particularly compact design of the nasal cannula.

According to one embodiment, the retaining band can be configured such that, during use of the nasal cannula, it covers at least 50%, at least 70% or at least 90% of a region of the head outside the face. In other words, the retaining band can be designed in the form of a cap in the ready-to-use state. Thus, the retaining band can additionally warm and/or protect the head similarly to a cap.

According to one embodiment, the retaining band can comprise a first band part for fastening the retaining band to the first cheek pad and a second band part, separate from the first band part, for fastening the retaining band to the second cheek pad and is designed such that the first band part can be connected to the second band part in different positions with respect to the retaining band in order to adapt the retaining band, in terms of its size and/or shape, to the head. It is possible for the retaining band to have an at least two-part design, an at least three-part design or an at least four-part design, wherein the retaining band can be designed such that the individual band parts can be connected to one another in a releasable manner. For example, the individual band parts can be adjustable in terms of their length and/or width. This allows particularly precise adaptation of the retaining band to different head sizes and/or shapes.

According to one embodiment, the retaining band can be designed such that the first band part can be connected to the second band part via a hook-and-loop connection in order to form the retaining band. This permits particularly secure and/or comfortable placement or removal of the retaining band. In addition, such a hook-and-loop connection can be very soft and/or flexible in comparison with other types of connection such as, for example, belt or buckle connections.

According to one embodiment, each of the band parts can comprise a cheek band section for fastening the band part to the respective cheek pad, a first headband section and a second headband section. In this case, the retaining band can be designed in such a way that the first headband sections of the different band parts can be connected to one another in different positions to form a first headband, in order to allow adaptation of a size and/or a shape of the first headband to the head, and the second headband sections of the different band parts can be connected to one another in different positions to form a second headband, in order to allow adaptation of a size and/or a shape of the second headband to the head. Furthermore, the retaining band can be designed in such a way that, during use of the nasal cannula:
the cheek band section fastened to the first cheek pad is tensioned around a first cheek region comprising at least part of the first cheek,
the cheek band section fastened to the second cheek pad is tensioned around a second cheek region, different from the first cheek region, comprising at least part of the second cheek,
the first headband is tensioned around a first head region comprising at least part of a parietal region of the head, and
the second headband is tensioned around a second head region, different from the first head region, comprising at least part of an occipital region and/or a neck region of the head.

This enables secure fixing of the nose attachment piece with respect to the face, even in very lively patients. Furthermore, the pressure exerted by the retaining band can in this way be distributed particularly uniformly over the head. Undesired pressure points can thus be avoided, even in the case of prolonged use of the nasal cannula.

Each of the cheek regions can additionally comprise at least part of the respective ear and/or of the respective temple of the patient.

For example, each of the headbands can have a different length and/or width in each of the different positions in which the respective headband sections can be connected to one another.

According to one embodiment, each of the band parts can further comprise a third headband section. In this case, the retaining band can be designed in such a way that the third headband sections of the different band parts can be connected to one another in different positions to form a third headband, in order to allow adaptation of a size and/or a shape of the third headband to the head.

Furthermore, the retaining band can be designed such that, during use of the nasal cannula, the third headband is tensioned around a third head region of the head that differs from the first head region and/or the second head region. For example, the third head region can comprise at least part of the neck region.

The retaining band can also comprise more than three such head bands.

According to one embodiment, the retaining band can be designed such that at least one of the headband sections of the first band part can be connected to the respective headband section of the second band part via a hook-and-loop connection in order to form the respective headband. This permits particularly secure and/or comfortable placement or removal of the respective headband. In addition, such a hook-and-loop connection can be very soft and/or flexible in comparison with other types of connection such as, for example, belt or buckle connections.

For example, the retaining band can be designed such that, during use of the nasal cannula, adjacent headbands are separated from one another in each case by a cutout, which exposes part of the head. This improves ventilation compared to an embodiment without such cutouts.

It is possible that each of the band sections, in a region in which the respective band section touches the head during use of the nasal cannula, has a length of at least 2 cm, preferably at least 4 cm. In this way, it is possible to avoid the individual band sections exerting excessive pressure on the particularly sensitive head.

It is also conceivable for each of the band parts to comprise a base section from which the respective band sections extend like fingers in different directions.

According to one embodiment, the nasal cannula can further comprise a plug connector by which a free end of the supply hose attached to the hose connector is connected to a respiratory gas source via a plug connector receptacle. The plug connector can be designed in such a way that it can be plugged together with the plug connector receptacle to form an airtight plug connection (and, if required, can be disconnected again). The plug connector, when plugged together with the plug connector receptacle, can be mounted on and/or in the plug connector receptacle in such a way that the plug connector and the plug connector receptacle can be rotated relative to each other about a common axis of rotation.

For example, the mounting can be designed in such a way that the plug connector and the plug connector receptacle can be rotated relative to each other by more than 360 degrees and/or in any desired directions of rotation. In this way, it is possible to avoid undesired twisting and/or buckling of hoses which are attached to the plug connection.

According to one embodiment, the nasal cannula can furthermore comprise the supply hose, wherein the supply hose is connected on the one hand to the hose connector and on the other hand to the plug connector.

According to one embodiment, the nasal cannula can furthermore comprise the plug connector receptacle and/or a connection hose for connecting the plug connector receptacle to the respiratory gas source. The connection hose can be, for example, a (spiral) hose that is particularly thick and/or stable in comparison with the supply hose described above.

According to one embodiment, the plug connector can comprise a tubular inner part, for conducting the respiratory gas, and a tubular outer part. The external diameter of the inner part can be smaller than the internal diameter of the outer part by a defined amount, and the inner part and the outer part can be arranged coaxially in such a way that the inner part and the outer part delimit a defined annular gap for receiving at least one section of the plug connector receptacle. The gap can be designed in such a way that the section of the plug connector receptacle received by the gap can be moved in the circumferential direction of the gap, in order to permit a rotation of the plug connector relative to the plug connector receptacle about a common central axis of the inner part and of the outer part as the common axis of rotation.

According to one embodiment, the plug connection can be a standardized plug connection, in particular according to DIN EN ISO 5356. The standard DIN EN ISO 5356 describes conical connectors for anesthesia and ventilation devices. Such connectors can be designed in the form of male and female cones (DIN EN ISO 5356-1) or in the form of weight-bearing connectors with screw threads (DIN EN ISO 5356-2).

According to one embodiment, the plug connector can have a fastening element for fastening the plug connector to an item of clothing or to a hose, for example. Such a fastening element can be designed, for example, as a clip and/or a bracket (similar to a ballpoint pen).

According to one embodiment, the nasal cannula can further comprise a hose holder for holding the supply hose in a defined position with respect to the face of the patient. The hose holder can for example be designed in such a way that it can be fastened to one of the cheek pads and/or to the retaining band in a releasable manner, for example by clamping, hook-and-loop fastening or adhesive bonding.

According to one embodiment, the nose attachment piece can furthermore comprise a further hose connector for connecting a further supply hose to the main body, such that respiratory gas can flow from the further supply hose into the main body. The hose connector and the further hose connector can be arranged, for example, at ends of the (elongate) main body lying opposite each other in the longitudinal direction of the main body.

According to one embodiment, the plug connector can be designed to furthermore connect a free end of the further supply hose, connected to the further hose connector, to the respiratory gas source via the plug connector receptacle.

In other words, the plug connector can have, on the one hand, an inlet connectable to the respiratory gas source and, on the other hand, an outlet connectable to the two supply hoses, wherein the inlet is fluidically coupled to the outlet. For example, the plug connector can have two separate outlets for the two supply hoses, such that the latter can be connected to the plug connector independently of each other.

According to one embodiment, the nasal cannula can furthermore comprise the further supply hose, wherein the further supply hose is connected on the one hand to the further hose connector and on the other hand to the plug connector.

According to one embodiment, the nasal cannula can furthermore comprise a pressure measurement port, for connecting a pressure-measuring device (for detecting an air pressure) to the nasal cannula, and a pressure measurement line. The pressure measurement line can be fluidically coupled to the pressure measurement port and, proceeding from the pressure measurement port, can extend through the main body into at least one of the nose pieces, in order to enable (air) pressure measurement in the nose, for example at the outlet of the nose piece or of the nose pieces. This enables very accurate oropharyngeal pressure measurement during high-flow therapy (see above). For example, a section of the pressure measurement line can extend from the pressure measurement port through the supply hose into the main body. In other words, the pressure measurement line can be at least partially integrated into the supply hose. This can simplify the handling of the nasal cannula. According to one embodiment, the pressure measurement port can be integrated into a housing of the plug connector, for example into the aforementioned inner and/or outer part. In other words, the plug connector can be designed such that it can be connected to the respiratory gas source and the pressure-measuring device at the same time. This has the advantage that a separate pressure measurement port can be dispensed with, which saves space and can simplify the handling of the nasal cannula.

### BRIEF DESCRIPTION OF THE DRAWINGS

Embodiments of the invention are described hereinafter with reference to the accompanying drawings. Neither the description nor the drawings should be understood as restricting the scope of the invention.

FIG. 1 shows a ventilation system according to an embodiment of the invention.

FIG. 2 shows a cross section through a plug connection from FIG. 1.

FIG. 3 shows a retaining band of a nasal cannula according to an embodiment of the invention with two headbands.

FIG. 4 shows a retaining band of a nasal cannula according to an embodiment of the invention with three headbands.

The figures are purely schematic and not true to scale. If identical reference signs are used in different drawings, then these reference signs designate identical or identically acting features.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

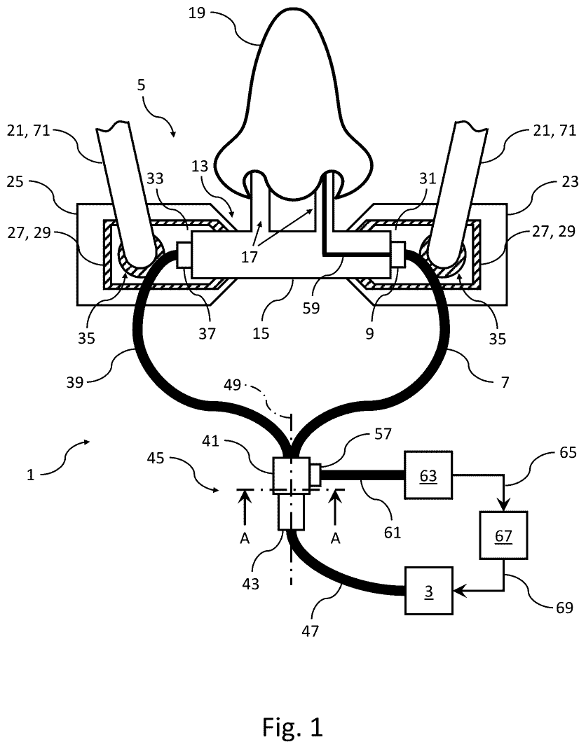

FIG. 1 shows a ventilation system 1 for ventilating a patient, here a premature or newborn baby or an infant, in the context of nasal high-flow therapy. The ventilation system 1 comprises a respiratory gas source 3 for providing a respiratory gas, for example oxygen or compressed air with increased oxygen concentration, a nasal cannula 5, and a supply hose 7, via which a hose connector 9 of the nasal cannula 5 is connected to the respiratory gas source 3, such that respiratory gas can flow from the respiratory gas source 3 via the supply hose 7 into the nasal cannula 5.

The nasal cannula 5 comprises a nose attachment piece 13 with a hollow, for example tubular or cylindrical, main body 15, and with two nose pieces 17 projecting from the main body 15 for insertion into the patient's nose 19. The nose attachment piece 13 is designed such that the respiratory gas can flow from the supply hose 7 via the hose connector 9 into the main body 15 and from there via the nose pieces 17 into the nostrils of the nose 19.

Depending on the embodiment, the nasal cannula 5 may also be suitable for at least one of the following patient types: children, young people, adults.

It is possible for at least one of the components of the nose attachment piece 13 to be produced at least partially from silicone and/or for the entire nose attachment piece 13 to be formed in one piece. It is also conceivable for the various components of the nose attachment piece 13 to be produced from the same material, for example silicone or another suitable plastic.

Furthermore, the nasal cannula 5 comprises a retaining band 21, which can be tensioned around the head of the patient, for fixing the nose attachment piece 13 in a defined position with respect to the face of the patient, for example by applying a corresponding additional retaining force to the nose attachment piece 13. The retaining band 21 can be embodied as an elongate element separate from the supply hose 7, for example as a rubber band and/or fabric band or as an arrangement of a plurality of such bands.

Furthermore, the nasal cannula 5 comprises, by way of example, a first cheek pad 23 and a second cheek pad 25 by which the nose attachment piece 13, fixed with respect to the face, is additionally supported and/or fixed on the two cheeks. The cheek pads 23, 25 can each be designed as a flat, in particular flexible element made of an air-permeable and/or liquid-absorbing material, for example a particularly skin-friendly and/or particularly soft textile material, and can touch the skin of the patient directly in a planar fashion during use of the nasal cannula 5. As a result, the contact pressure generated by the retaining band 21 tensioned around the head can be distributed uniformly over the largest possible surface area of the face, in particular of the cheeks. Undesired pressure points on the face can thus be avoided.

The nasal cannula 5 can be designed in such a way that the nose attachment piece 13 can be fastened to the retaining band 21 on the one hand via the first cheek pad 23 and on the other hand via the second cheek pad 25, for example via one or more hook-and-loop and/or adhesive connections.

In this example, each of the cheek pads 23, 25, has, on a first side facing the respective cheek during use of the nasal cannula 5, a skin-friendly contact section for contacting the respective cheek and, on a second side opposite the first side and facing away from the respective cheek during use of the nasal cannula 5, a fastening section 27. Here, the fastening section 27 comprises an adhesive portion and/or fleece portion 29 of a hook-and-loop connection for fastening the cheek pad 23 or 25 to the retaining band 21 and/or to the nose attachment piece 13. Alternatively or additionally, the fastening section 27 may for this purpose comprise a (self-adhesive) adhesion section.

The contact section may be at least partially composed of silicone and/or comprise a specific adhesive surface for adhesively bonding the respective cheek pad 23 or 25 to the respective cheek.

It is possible for each of the cheek pads 23, 25 to be formed as a layer composite composed of at least a first layer and a second layer, wherein the layers can differ from one another in terms of their material and/or their size (for example their thickness) and/or their shape. Here, the contact section can be a section of the first layer, and/or the fastening section 27 can be a section of the second layer. The second layer can be formed as a carrier for stabilizing the first layer (or vice versa). For example, the carrier can be produced from a particularly air-permeable textile material. However, other carrier materials are also possible.

In addition, the nose attachment piece 13 comprises a first fastening wing 31 projecting from the main body 15, for fastening the first cheek pad 23 to the nose attachment piece 13, and a second fastening wing 33 projecting from the main body 15, for fastening the second cheek pad 25 to the nose attachment piece 13. Here, the fastening section 27 of the first cheek pad 23 can be fastened to the first fastening wing 31, or the fastening section 27 of the second cheek pad 25 can be fastened to the second fastening wing 33 by means of a hook-and-loop fastener.

The fastening wings 31, 33 can in each case be connected to the main body 15 in a fixed or releasable manner, for example via a hook-and-loop and/or adhesively bonded connection. It is possible for the fastening wings 31, 33 to be particularly flexible in comparison with the main body 15, such that they can adapt well to the facial contours of the patient. In particular, the fastening wings 31, 33 can be produced from a for example particularly air-permeable textile material and/or have a fleecy surface at least in parts. It is also conceivable for the fastening wings 31, 33 and the main body 15 to be formed in one piece and/or from the same material.

It is possible for each of the fastening wings 31, 33 to have a through-opening 35 which connects a top side of the fastening wing 31 or 33 to the respective bottom side thereof. The opening 35 can in each case be arranged such that it lies at least partially opposite the respective fastening section 27 of the cheek pad 23 or 25 fastened to the respective fastening wing 31 or 33. It is thus possible for the retaining band 21 to be fastened to the parts of the fastening sections 27 opposite the openings 35, in this case for example by means of a hook-and-loop fastener.

In this example, the main body 15 additionally comprises a further hose connector 37, which can be connected to the respiratory gas source 3 via a further supply hose 39, such that the respiratory gas can additionally flow from the further supply hose 39 into the main body 15 and from there via the nose pieces 17 into the nose 19.

The free ends of the two supply hoses 7, 39 can in each case be connected to a plug connector 41 which can be plugged together with a corresponding plug connector receptacle 43 in order to form an airtight plug connection 45, which can be released again with a certain expenditure of force by pulling apart the plug connector 41 and the plug connector receptacle 43. The plug connector receptacle 43 can be connected to the respiratory gas source 3 via a connection hose 47, such that the respiratory gas can flow into the two supply hoses 7, 39 via the connection hose 47 and the plug connection 45.

The plug connection 45 can optionally be embodied as a rotary connection which allows targeted rotation of the plug connector 41 relative to the plug connector receptacle 43 about a common axis of rotation 49, for example by more than 360 degrees and/or in any desired directions of rotation.

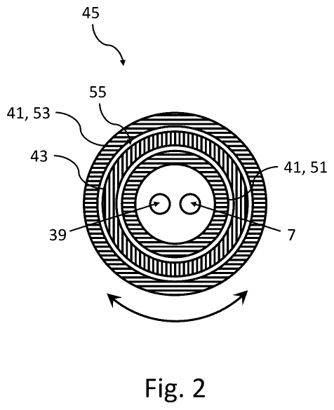

As can be seen from FIG. 2, where a cross section through the plug connection 41 from FIG. 1 is shown along a section line A-A, the plug connector 41 can for this purpose be embodied, for example, with a tubular or sleeve-like inner part 51 for conducting the respiratory gas and with a tubular or sleeve-like outer part 53. Here, the external diameter of the inner part 51 can be smaller than the internal diameter of the outer part 53 by a defined amount, and the inner part 51 and the outer part 53 can be arranged coaxially in such a way that they delimit a defined annular gap 55 for mounting the plug connector receptacle 43. The gap 55 can be formed in such a way that it can receive at least one section of the plug connector receptacle 43, and the section of the plug connector receptacle 43 received by the gap 55 can be rotated in both directions of rotation relative to the inner part 51 or to the outer part 53 with little expenditure of force. In this example, the axis of rotation 49 coincides with a common central axis of the inner part 51 and of the outer part 53.

The free ends of the two supply hoses 7, 39, can be connected, for example, to a corresponding connection or corresponding (separate) connections of the inner part 51.

In particular, the plug connection 45 can be embodied as a standardized plug connection, for example according to DIN EN ISO 5356-1 or 5356-2.

As is shown in FIG. 1, the nasal cannula 5 can additionally comprise a pressure measurement port 57, and a pressure measurement line 59 connected to the pressure measurement port 57. The pressure measurement line 59 can extend from the pressure measurement port 57 into at least one of the two nose pieces 17. This enables an oropharyngeal pressure measurement during nasal high-flow therapy.

In this example, the pressure measurement port 57 is integrated into a housing of the plug connector 41. Here, the pressure measurement line 59 extends through the supply hose 7 and the main body 15 as far as the open end of the right-hand one of the two nose pieces 17.

The pressure measurement port 57 can be connected to a pressure-measuring device 63 of the ventilation system 1 via a further connection hose 61. The pressure-measuring device 63 can be designed to convert a pneumatic pressure present at the pressure measurement port 57 into a corresponding electrical pressure measurement signal 65.

The pressure measurement signal 65 can be used by a correspondingly configured control unit 67 of the ventilation system 1 to control at least one ventilation parameter of the high-flow therapy. Depending on the embodiment, such a ventilation parameter may be a respiratory gas pressure, a respiratory gas flow, a respiratory gas volume or a respiratory gas composition. For example, the control unit 67 can be configured to generate a control signal 69 for controlling at least one actuator of the respiratory gas source 3, e.g. a blower or a valve, depending on the oropharyngeal pressure measured in this way.

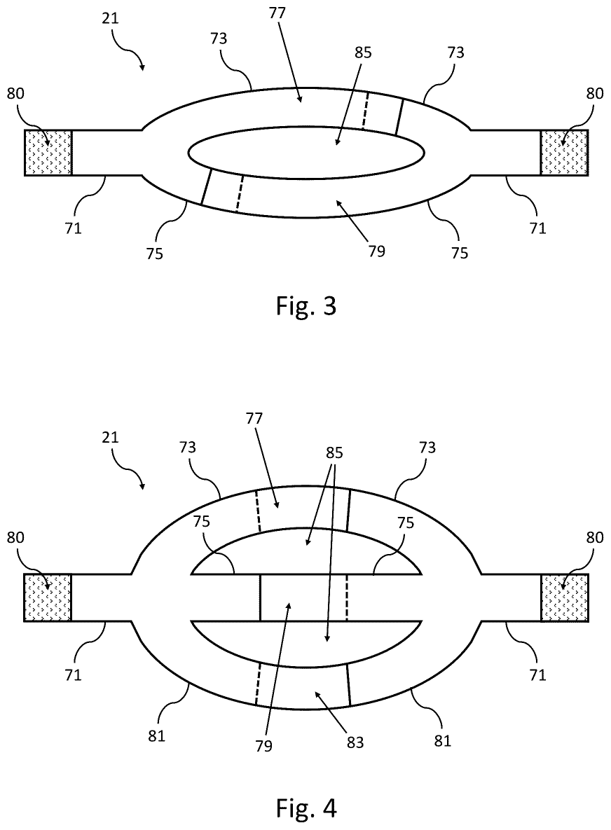

As can be seen from FIG. 3 and FIG. 4, the retaining band 21 can comprise a first band part for fastening the retaining band 21 to the first cheek pad 23, and a second band part, separate from the first band part, for fastening the retaining band 21 to the second cheek pad 25. The two band parts can be connected to each other in different positions—for example via one or more hook-and-loop fasteners—in order to form a retaining band 21 adapted to the respective head size or shape.

FIG. 3 shows an embodiment in which each of the band parts comprises a cheek band section 71 for fastening the band part to the respective cheek pad 23 or 25, a first headband section 73 and a second headband section 75. The first headband sections 73 of the different band parts can be connected to one another in different positions in order to form a first headband 77 adapted to the respective head size or shape. Likewise, the second headband sections 75 of the different band parts can be connected to one another in different positions in order to form a second headband 79 adapted to the respective head size or shape.

The retaining band 21 can be configured such that, during use of the nasal cannula 5, the cheek band section 71 fastened to the first cheek pad 23 is tensioned around a first cheek region comprising at least part of the first cheek, the cheek band section 71 fastened to the second cheek pad 25 is tensioned around a second cheek region that differs from the first cheek region and that comprises at least part of the second cheek, the first headband 77 is tensioned around a first head region comprising at least part of a parietal region of the head, and the second headband 79 is tensioned around a second head region that differs from the first head region and comprises at least part of an occipital region and/or a neck region of the head.

By way of example, the cheek band sections 71 can each have, at their free ends, a further adhesive portion and/or fleece portion 80, which can be connected to the adhesive part or fleece portion 29 of the respective fastening section 27 in order to form the respective hook-and-loop connection.

Each of the cheek regions can additionally comprise at least part of the respective ear and/or of the respective temple of the patient.

FIG. 4 shows an embodiment in which each of the band parts additionally comprises a third headband section 81. Analogously to the headband sections 73, 75 described above, the third headband sections 81 of the different band parts can be connected to one another in different positions in order to form a third headband 83 adapted to the respective head size and/or shape, and, during use of the nasal cannula 5, the third headband is tensioned around a third region of the head distinct from the first head region and/or the second head region. For example, the third head region can comprise at least part of the neck region.

The retaining band 21 can also comprise more than three such head bands.

For example, each of the headbands can have a different length and/or width in each of the different positions in which the respective headband sections can be connected to one another.

It is possible for each of the band sections to have a width and/or length of at least 2 cm, preferably at least 4 cm, in a region in which the respective band section touches the head during use of the nasal cannula 5. In this way, it is possible to avoid the individual band sections exerting excessive pressure on the particularly sensitive head.

The retaining band 21 can also be embodied like a cap such that, during use of the nasal cannula 5, it covers at least 50%, at least 70% or at least 90% of a region of the head outside the face.

It is possible that, during use of the nasal cannula 5, adjacent headbands are separated from one another in each case by a cutout 85, which exposes part of the head. This improves ventilation compared to an embodiment without such cutouts.

The respective headband sections can be connectable to one another, for example, by means of a hook-and-loop fastener.

Finally, it will be noted that terms such as “have”, “comprise”, “include”, “with” etc. do not exclude other elements or steps, and indefinite articles such as “a” or “an” do not exclude a plurality.

Furthermore, it will be noted that features or steps described with reference to one of the embodiments above can also be used in combination with features or steps described with reference to other embodiments from among the embodiments above.

Reference signs in the claims should not be understood as restricting the scope of the subject matter defined by the claims.

### LIST OF REFERENCE SIGNS

1 ventilation system
3 respiratory gas source
5 nasal cannula
7 supply hose
9 hose connector
13 nose attachment piece
15 main body
17 nose piece
19 nose
21 retaining band
23 first cheek pad
25 second cheek pad
27 fastening section
29 adhesive portion and/or fleece portion
31 first fastening wing
33 second fastening wing
35 opening
37 further hose connector
39 further supply hose
41 plug connector
43 plug connector receptacle
45 plug connection
47 connection hose
49 axis of rotation, central axis
51 inner part
53 outer part
55 gap
57 pressure measurement port
59 pressure measurement line
61 further connection hose
63 pressure-measuring device
65 pressure measurement signal
67 control unit
69 control signal
71 cheek band section
73 first headband section
75 second headband section
77 first headband
79 second headband
80 further adhesive portion and/or fleece portion
81 third headband section
83 third headband
85 cutout

What is claimed is:

1. A nasal cannula for supplying respiratory gas to a patient in the context of high-flow therapy, wherein the patient is a premature or newborn baby or an infant, wherein the nasal cannula comprises:
a nose attachment piece, which has a hollow main body, a hose connector for connecting a supply hose to the main body, such that respiratory gas can flow from the supply hose into the main body, and two nose pieces projecting from the main body, for conducting the respiratory gas from the main body into a nose of the patient;
a first cheek pad for supporting and/or fixing the nose attachment piece on a first cheek of the patient;
a second cheek pad for supporting and/or fixing the nose attachment piece on a second cheek of the patient;
a retaining band, which can be tensioned around the head of the patient, for applying an additional retaining force to the nose attachment piece and/or the two cheek pads.

2. The nasal cannula of claim 1,
wherein the nasal cannula is designed such that the nose attachment piece can be fastened to the retaining band on the one hand via the first cheek pad and on the other hand via the second cheek pad; and/or
wherein each of the first and second cheek pads can be fastened to the retaining band and/or to the nose attachment piece via a hook-and-loop connection.

3. The nasal cannula of claim 1,
wherein each of the first and second cheek pads has, on a first side facing the respective cheek during use of the nasal cannula, a skin-friendly contact section for contacting the respective cheek and, on a second side opposite the first side, a fastening section which comprises an adhesive portion and/or a fleece portion of a hook-and-loop connection for fastening the cheek pad to the retaining band and/or to the nose attachment piece.

4. The nasal cannula of claim 1, wherein the nose attachment piece further comprises:
a first fastening wing projecting from the main body, for fastening the first cheek pad to the nose attachment piece;
a second fastening wing projecting from the main body, for fastening the second cheek pad to the nose attachment piece.

5. The nasal cannula of claim 4,
wherein, for fastening the first cheek pad to the nose attachment piece, the fastening section of the first cheek pad is fastenable to the first fastening wing and, for fastening the second cheek pad to the nose attachment piece, the fastening section of the second cheek pad is fastenable to the second fastening wing.

6. The nasal cannula of claim 5,
wherein the first fastening wing has a through-opening which connects a top side of the first fastening wing to a bottom side of the first fastening wing opposite the top side and is arranged such that the fastening section of the first cheek pad fastened to the first fastening wing is at least partially opposite the opening, such that the retaining band can be fastened to the fastening section of the first cheek pad opposite the opening; and
wherein the second fastening wing has a through-opening which connects a top side of the second fastening wing to a bottom side of the second fastening wing opposite the top side and is arranged such that the fastening section of the second cheek pad fastened to the second fastening wing is at least partially opposite the opening, such that the retaining band can be fastened to the fastening section of the second cheek pad opposite the opening.

7. The nasal cannula of claim 1,
wherein the retaining band is designed such that, during use of the nasal cannula, it covers at least 50% of a region of the head outside the face; and/or
wherein the retaining band comprises a first band part for fastening the retaining band to the first cheek pad and a second band part, separate from the first band part, for fastening the retaining band to the second cheek pad and is designed such that the first band part can be connected to the second band part in different positions with respect to the retaining band in order to adapt the retaining band, in terms of its size and/or shape, to the head.

8. The nasal cannula of claim 7,
wherein each of the band parts comprises a cheek band section for fastening the band part to the respective cheek pad, a first headband section and a second headband section;
wherein the first headband sections of the different band parts are connectable to one another in different positions to form a first headband, in order to adapt the size and/or shape of the first headband to a head;
wherein the second headband sections of the different band parts are connectable to one another in different positions to form a second headband, in order to adapt the size and/or shape of the second headband to the head;
wherein the retaining band is designed such that, during use of the nasal cannula, the cheek band section fastened to the first cheek pad is tensioned around a first cheek region comprising at least part of the first cheek, the cheek band section fastened to the second cheek pad is tensioned around a second cheek region that differs from the first cheek region and that comprises at least part of the second cheek, the first headband is tensioned around a first head region comprising at least part of a parietal region of the head, and the second headband is tensioned around a second head region that differs from the first head region and that comprises at least part of an occipital region and/or a neck region of the head.

9. The nasal cannula of claim 8,
wherein each of the band parts furthermore comprises a third headband section;
wherein the third headband sections of the different band parts are connectable to one another in different positions to form a third headband, in order to adapt the size and/or shape of the third headband to the head;
wherein the retaining band is designed such that, during use of the nasal cannula, the third headband is tensioned around a third head region of the head that differs from the first head region and/or the second head region.

10. The nasal cannula of claim 8,
wherein at least one of the headband sections of the first band part can be connected to the respective headband section of the second band part via a hook-and-loop connection in order to form a respective headband.

11. The nasal cannula of claim 1, wherein the cannula further comprises:
a pressure measurement port for connecting a pressure-measuring device to the nasal cannula;
a pressure measurement line, which is fluidically coupled to the pressure measurement port and, proceeding from the pressure measurement port, extends through the main body into at least one of the nose pieces in order to enable a pressure measurement in the nose.

12. A nasal cannula for supplying respiratory gas to a patient in the context of high-flow therapy, wherein the nasal cannula comprises:
a nose attachment piece, which has a hollow main body, a hose connector for connecting a supply hose to the main body, such that respiratory gas can flow from the supply hose into the main body, and two nose pieces projecting from the main body, for conducting the respiratory gas from the main body into a nose of the patient;
a pressure measurement port for connecting a pressure-measuring device to the nasal cannula;
a pressure measurement line, which is fluidically coupled to the pressure measurement port and, proceeding from the pressure measurement port, extends through the main body into at least one of the nose pieces in order to enable a pressure measurement in the nose.

13. The nasal cannula of claim 12, wherein the cannula further comprises:
a plug connector for connecting a free end of the supply hose, connected to the hose connector, to a respiratory gas source via a plug connector receptacle, wherein the plug connector is designed such that the plug connector can be plugged together with the plug connector receptacle to form an airtight plug connection, wherein the plug connector, when plugged together with the plug connector receptacle, is mounted on and/or in the plug connector receptacle in such a way that the plug connector and the plug connector receptacle can be rotated relative to each other about a common axis of rotation.

14. The nasal cannula of claim 13,
wherein the pressure measurement port is integrated into a housing of the plug connector.

15. A ventilation system, wherein the system comprises:
the nasal cannula of claim 12;
a supply hose for connecting the hose connector of the nasal cannula to a respiratory gas source, such that respiratory gas can flow from the respiratory gas source via the supply hose into the main body of the nasal cannula;
a pressure-measuring device connected to the pressure measurement port of the nasal cannula, for converting a pressure present at the pressure measurement port into an electrical pressure measurement signal
a control unit for controlling at least one ventilation parameter of the high-flow therapy using the pressure measurement signal.

16. A ventilation system, wherein the system comprises:
the nasal cannula of claim 11;
a supply hose for connecting the hose connector of the nasal cannula to a respiratory gas source, such that respiratory gas can flow from the respiratory gas source via the supply hose into the main body of the nasal cannula;
a pressure-measuring device connected to the pressure measurement port of the nasal cannula, for converting a pressure present at the pressure measurement port into an electrical pressure measurement signal
a control unit for controlling at least one ventilation parameter of the high-flow therapy using the pressure measurement signal.

17. The nasal cannula of claim 1, wherein the retaining band is designed such that, during use of the nasal cannula, it covers at least 60% of a region of the head outside the face.

18. The nasal cannula of claim 1, wherein the retaining band is designed such that, during use of the nasal cannula, it covers at least 70% of a region of the head outside the face.

19. The nasal cannula of claim 1, wherein the retaining band is designed such that, during use of the nasal cannula, it covers at least 90% of a region of the head outside the face.
